(19) United States
(12) Patent Application Publication
(10) Pub. No.: US 2018/0064399 A1
(43) Pub. Date: Mar. 8, 2018

(54) IMAGING SYSTEMS INCLUDING MULTI-TAP DEMODULATION PIXELS FOR BIOMETRIC MEASUREMENTS

(71) Applicant: Heptagon Micro Optics Pte. Ltd., Singapore (SG)

(72) Inventors: Bernhard Buettgen, Adliswil (CH); Jens Geiger, Thalwil (CH); Michael Kiy, Winterthur (CH); Oliver Chidley, Regensdorf (CH); Markus Rossi, Jona (CH)

(73) Assignee: Heptagon Micro Optics Pte. Ltd., Singapore (SG)

(21) Appl. No.: 15/696,431

(22) Filed: Sep. 6, 2017

Related U.S. Application Data
(60) Provisional application No. 62/384,223, filed on Sep. 7, 2016.

(51) Int. Cl. A61B 5/00; G01S 17/88; G01S 7/491; H04N 5/378; G06K 9/00; A61B 5/024; A61B 5/1455

(52) U.S. Cl. CPC A61B 5/7228; G01S 17/88; G01S 7/4915; H04N 5/378; G06K 2009/00939; A61B 5/02433; A61B 5/14551; A61B 5/02416; A61B 5/0077; G06K 9/00885

(57) ABSTRACT

Imaging systems include multi-tap demodulation pixels for biometric measurements such as heart rate or blood oxygen level. Using multi-tap demodulation pixels can, in some cases, help facilitate the generation of differential signals to remove background noise and achieve a higher dynamic range for the biometric measurements.

### CROSS-REFERENCE TO RELATED APPLICATION(S)

This application claims the benefit of U.S. Provisional Patent Application No. 62/384,223, filed on Sep. 7, 2016. The contents of the earlier application are incorporated herein by reference in their entirety.

### TECHNICAL FIELD

This disclosure relates to imaging systems including multi-tap demodulation pixels for biometric measurements.

### BACKGROUND

Various types of sensors are used widely in physics, chemistry, and engineering and find use in a wide range of applications. Some of these sensors use optical signals to measure parameters of interest, e.g., pressure, distance, temperature or composition.

In some cases, modules incorporating such sensors are used in medical and health-related applications. For example, performing a measurement on a human body can include bringing a portion of the human body into proximity with the module, directing light emitted from the module toward the portion of the human body, and detecting light reflected by the portion of the human body into the module. Information based on the light detected by the module can be processed, for example, to provide an indication of a physical condition of the human body.

### SUMMARY

This disclosure describes imaging systems including multi-tap demodulation pixels for biometric measurements.

In one aspect, for example, the disclosure describes an optoelectronic module that includes a controller, an illumination source, and a TOF image sensor. The illumination source is operable to emit radiation of a particular wavelength toward a subject outside the module. The TOF image sensor includes one or more multi-tap demodulation pixels operable to sense radiation of the particular wavelength reflected by the subject back toward the module. Each of the one or more demodulation pixels includes a plurality of charge storage nodes, and a read-out circuit to provide signals to the controller. The controller is operable to temporally modulate and synchronize the TOF image sensor and the illumination source, and to determine a biometric characteristic of the subject based at least in part on the signals from the one or more demodulation pixels.

Some implementations include one or more of the following features. For example, in some instances, each of the one or more demodulation pixels has a 2-tap configuration. In some cases, each of the one or more demodulation pixels includes first and second charge storage nodes, and an in-pixel circuit to generate a differential signal based on charges stored in the pixel's first and second charge storage nodes. The read-out circuit can be operable to provide the differential signal to the controller, which can be operable to temporally modulate and synchronize the TOF image sensor and the illumination source such that only the first storage node of each TOF pixel integrates photo-generated electrons while the illumination source is turned on, and only the second storage node of each TOF pixel integrates photo-generated electrons when the illumination source is turned off.

In some implementations, the biometric characteristic is a heart rate of the subject. The module can be placed, for example, adjacent the subject such that when at least a portion of the radiation emitted by the illumination source passes into, and is reflected by, the subject, at least some of the reflected radiation is sensed by the demodulation pixels. In some instances, the illumination source is operable to emit infra-red radiation.

In a related aspect, the disclosure also describes a method of operating an optoelectronic module. The method includes emitting radiation, having a particular wavelength, from an illumination source in the module toward a subject, sensing, by one or more multi-tap TOF demodulation pixels in the module, radiation of the particular wavelength reflected by the subject back toward the module, and determining a biometric characteristic of the subject based at least in part on signals from the one or more demodulation pixels.

In accordance with another aspect, the disclosure describes an optoelectronic module that includes a controller; first and second illumination sources, and an imager sensor. The first illumination source is operable to emit radiation at a first wavelength toward a subject outside the module, and the second illumination source is operable to emit radiation at a second wavelength different from the first wavelength toward the subject. The image sensor includes one or more multi-tap demodulation pixels, each of which is operable to sense radiation of the first wavelength reflected by the subject back toward the module and to sense radiation of the second wavelength reflected by the subject back toward the module. Each of the one or more multi-tap demodulation pixels includes a plurality of charge storage nodes, and a read-out circuit to provide signals to the controller. The controller is operable to temporally modulate and synchronize the image sensor and the illumination sources, and to determine a biometric characteristic of the subject based at least in part on the signals from the demodulation pixels.

Some implementations include one or more of the following features. For example, in some cases, each of the demodulation pixels has a 2-tap configuration. In some implementations, each of one or more demodulation pixels includes first and second charge storage nodes, and an in-pixel circuit to generate a differential signal based on charges stored in the pixel's first and second charge storage nodes. The read-out circuit is operable to provide the differential signal to the controller, which is operable to temporally modulate and synchronize the image sensor and the illumination sources such that only one of the first or second illumination sources is turned on at a given time. Photo-generated charges resulting from back-reflected radiation of the first illumination source are transferred to the respective first charge storage node of each of the one or more demodulation pixels, and photo-generated charges resulting from back-reflected radiation of the second illumination source are transferred to the respective second charge storage node of each of the one or more demodulation pixels.

In some instances, the first illumination unit is operable to emit infra-red radiation, and the second illumination unit is operable to emit visible radiation. In some cases, the module includes a respective dual band-pass filter over each of the one or more demodulation pixels. The dual band-pass filter is operable selectively to pass radiation of the first wavelength and the second wavelength.

In some implementations, the biometric characteristic is an oxygen saturation level of the blood of the subject.

In a related aspect, the disclosure describes a method of operating an optoelectronic module. The method includes emitting radiation of a first wavelength from a first illumination source in the module toward a subject, and sensing, by a multi-tap demodulation pixel in the module, radiation of the first wavelength reflected by the subject back toward the module. The method further includes emitting radiation of a second wavelength from a second illumination source in the module toward the subject, and sensing, by the multi-tap demodulation pixel in the module, radiation of the second wavelength reflected by the subject back toward the module. A biometric characteristic of the subject is determined based at least in part on a signal from the multi-tap demodulation pixel.

Various advantages are present in some implementations. For example, in some cases, use of the multi-tap demodulation pixels can help increase the dynamic range for optical-based biometric measurements such as heart rate and blood oxygen, among others.

Other aspects, features and advantages will be readily apparent from the following detailed description, the accompanying drawings and the claims.

### BRIEF DESCRIPTION OF THE DRAWINGS

FIG. 1A illustrates an example of a time-of-flight (“TOF”) demodulation pixel.

FIG. 1B is a timing diagram of a 2-tap configuration.

FIGS. 1C and 1D are time zoom diagrams of FIG. 1B.

FIG. 2 illustrates an example of a module including an image sensor having multi-tap demodulation pixels.

FIG. 3 illustrates use of the module of FIG. 2 for heart rate measurements.

FIG. 4 illustrates another example of a module including an image sensor having multi-tap demodulation pixels.

### DETAILED DESCRIPTION

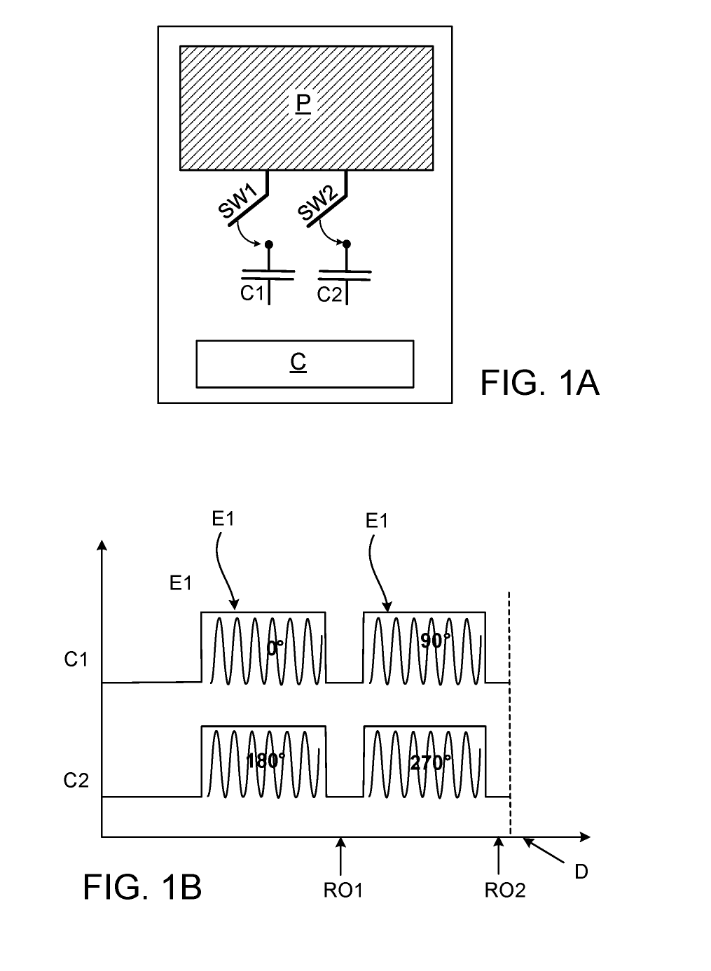

As shown in the example of FIG. 1A, a time-of-flight (“TOF”) demodulation pixel includes a photo-sensitive area P, connected by a first switch SW1 to a first storage node C1, and connected by a second switch SW2 to second storage node C2. Sampling of the photo-generated electrons is accomplished by closing switch SW1 and opening SW2, or vice-versa. The switches SW1 and SW2 are synchronized to one or more illumination sources by a controller. To enable reasonable time of flight measurements, the switches SW1, SW2 and illumination sources should operate in the range of around 10 MHz to 200 MHz (or higher), and the photo-generated charges should be transferred from the photo-sensitive area P to either storage node C1 or C2 within a few nanoseconds (“ns”). TOF pixels are specifically designed to reach such high-speed samplings. Examples of such high-speed TOF pixel architectures are described in the following patents: U.S. Pat. No. 5,856,667, EP 1009984B1, EP 1513202B1 and U.S. Pat. No. 7,884,310, which are incorporated herein by reference.

Output circuitry C of the TOF pixel can include a readout amplifier and a reset node. In some TOF pixel implementations, the in-pixel output circuitry C further includes a common level removal or background subtraction circuitry of the two samples in the storage nodes C1, C2. Such in-pixel common signal level removal can increase the dynamic range of the TOF pixels. Example implementations that perform a common level removal of the samples while integrating the signals are described in PCT publication WO 2009/135952A2 and in U.S. Pat. No. 7,574,190. In some cases, common signal level subtraction of the samples is performed after exposure during the readout cycle of the data as described, for example, in U.S. Pat. No. 7,897,928. The disclosures of these documents is incorporated herein by reference.

Some three-dimensional TOF measurement systems are based either on sine wave or pseudo-noise modulation and require at least three samples to derive phase or depth information respectively, offset, and amplitude information. For design simplification and signal robustness reasons, TOF systems commonly sample the impinging light signal four times. Nevertheless, highly sensitive TOF pixel architectures include only two storage nodes, as illustrated in the 2-tap implementation of FIGS. 1A and 1B. To obtain four samples, the system acquires at least two images. The received light signal generates a photo-current, which then is sampled and integrated during a first exposure E1 and subsequently during a second exposure, E2 as shown in FIG. 1B. A time zoom in the sampling of the first exposure E1 is shown in FIG. 1C; a time zoom in the sampling of the second exposure E2 is shown in FIG. 1D.

In the illustrated 2-tap example, the first exposure E1 is sampled at 0° and 180°, and is followed by a readout RO1. A second exposure E2 is sampled with a delay of 90° relative to the first exposure E1 (i.e., samples at 90° and 270°). After the second exposure E2, the newly acquired samples are readout RO2. The sampling duration is assumed to be half of the period and can be integrated over many thousands or millions of periods. Integration times of exposure E1 and exposure E2 should be substantially the same. During the first exposure E1, the samples at 0° and 180° are directed by the switches SW1 and SW2 to the storage nodes C1 and C2, respectively. During the second exposure, the samples at 90° and 270° are directed by the switches SW1 and SW2 to the storage nodes C1 and C2, respectively. When all four samples have been measured and are available (e.g., at time D), the phase, amplitude and offset information can be calculated; the phase information corresponds directly to the distance information of the measured object.

As a result of mismatches, some implementations acquire four, instead of only two, images. In such cases, the first and second exposures E1, E2 and their readouts RO1, RO2 can be performed as described with respect to FIG. 1B, but these exposures are followed by two more exposures E3, E4 and readouts RO3, RO4. Exposure E3 acquires the samples the first exposure E1, but delayed by 180°; likewise, the exposure E4 corresponds to a 180° phase delay of exposure E2. All four samples then are available to calculate phase (or depth respectively), offset and amplitude information of the received signal.

Other multi-tap implementations are possible as well. For example, in some implementations, four samples are acquired in parallel (e.g., at 0°, 90°, 180° and 270°) during a single exposure and then are read out (“4-tap”).

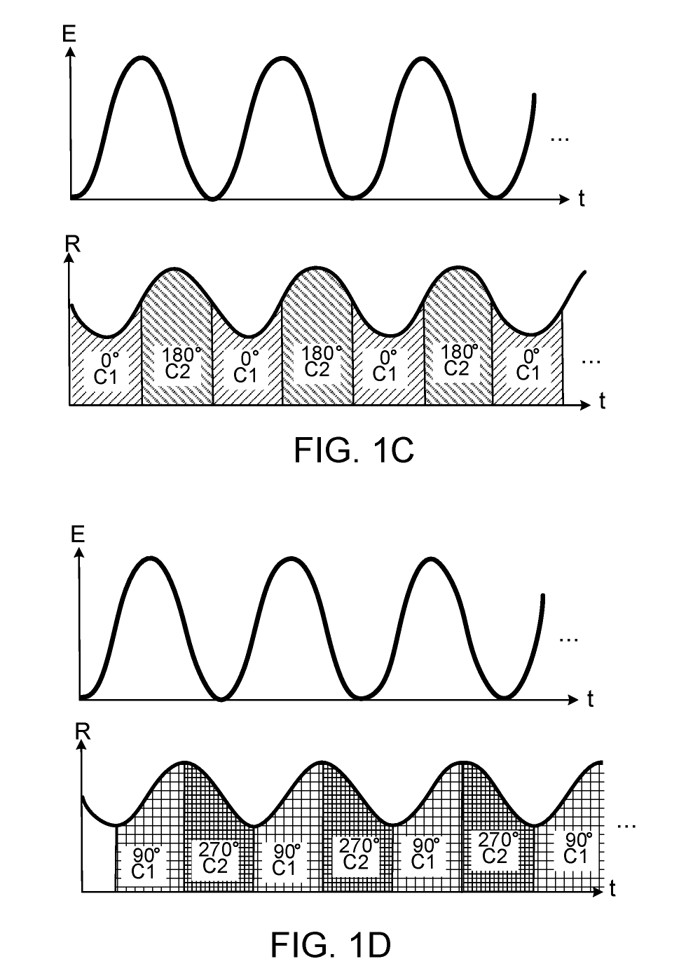

As illustrated in FIG. 2, an imaging module 100 includes a TOF image sensor 110 having multi-tap TOF demodulation pixels as described above (e.g., a 2-tap or other multi-tap configuration), an illumination source 120, an optical system 130 and a controller 140. Emitted light 120a is reflected by an object 10, and the back-reflected light 120b is imaged by the optical system 130 onto the TOF image sensor 110. The TOF image sensor 110 and the illumination source 120 are temporally modulated and synchronized by the controller 140 such that a first one of the storage nodes of each TOF pixel on the image sensor 110 integrates all photo-generated electrons while the illumination source 120 is turned on, and a second storage node of each TOF pixel integrates all photo-generated electrons when the illumination source 120 is turned off. This on/off cycle can be repeated multiple times. In some instances, in-pixel circuitry is operable to perform a direct subtraction of the two nodes during integration and by doing so removes the common background light signal. In other implementations, the in-pixel circuitry performs the subtraction at the end of the integration time. Both implementations can help increase the dynamic range of the active imaging module. To achieve good background light removal, the total exposure time into the two storage nodes should be the same. After the exposure, the pixel values are read out and transferred to the controller 140. Before starting the next acquisition, the pixels can be reset RS.

As described below, the imaging module 100 can be implemented as a biometric sensor such as a heart rate monitor that acquires measurements of an individual's heart rate. In such implementations, the illumination source 120 preferably is operable to emit light in the infra-red (“IR”) part of the spectrum (e.g., 850 nm-950 nm). The illumination source 120 can be implemented, for example, as a light emitting diode (LED), an organic LED (OLED), a laser or a vertical cavity surface emitting laser (VCSEL).

### Heart Rate Measurement Sensor Module

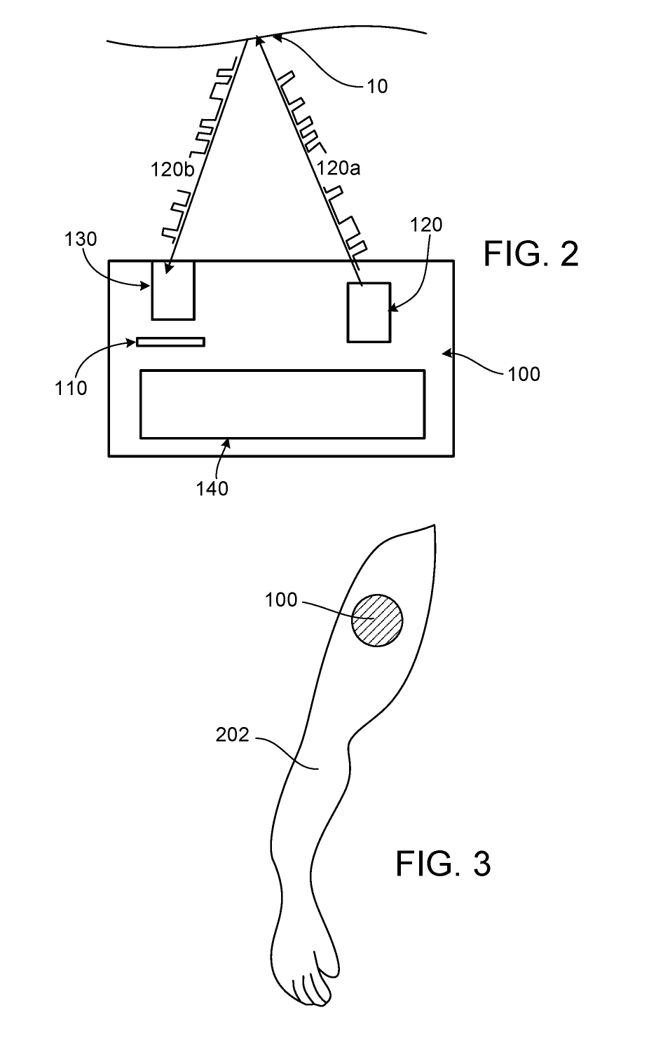

In operation, as illustrated in FIG. 3, the imaging module 100 is placed against the skin of a subject (e.g., on a limb of a user) 202. During operation, the module 100 emits IR light onto the skin of the subject 202. A portion of the light passes through the skin into the subcutaneous tissue where it may encounter blood vessels carrying oxygenated arterial blood. With each cardiac cycle, the heart pumps blood through such vessels, causing the blood vessels to expand. The expansion and contraction of the blood vessels and the variation in the amount of oxygenated hemoglobin with each cycle modulates the light reaching the module 100 and sensed by the demodulation pixels. Heart rate measurements can be obtained by using the multi-tap demodulation pixel(s) to account for background light and increase dynamic range. In particular, while the illumination source 120 is on (i.e., emitting IR radiation), the photo-generated charges are collected in the pixel's first storage node (i.e., tap 1). The, while the illumination source 120 is off (i.e., non-emitting state), the photo-generated charges are collected in the pixels' second storage node (i.e., tap 2).

Next, the in-pixel circuitry subtracts the charge collected by the second storage node from the charge collected by the first storage node so as to generate a differential signal. The in-pixel circuitry to perform the subtraction can be implemented in various ways. For example, as described in PCT published patent application WO 2010/144616, a third common storage node (e.g., a capacitance) can be provided to subtract the two demodulated charge packets on the storage nodes C1, C2. This third storage node is separated from the demodulation pixel and the sampling storage nodes C1, C2 by switches controlled by a voltage pattern generator. The disclosure of WO 2010/144616 is incorporated herein by reference. In some implementations, other in-pixel circuitry is provided for the subtraction.

The resulting differential signal(s) are read out by a read-out circuit to the control circuit 140. By monitoring the time-varying change in the amount of IR light reflected back toward, and sensed by, the module 100, the control circuit 140 can calculate the corresponding heart rate of the subject 102. The foregoing technique can lead to high dynamic range and efficient background light suppression for heart rate measurements and monitoring.

In some implementations, the module 100 includes a carrier substrate such as an adhesive pad so that the module 100 can be affixed to the subject or it may include a strap, band, bracelet, watchband, tape, or other structure that can be fitted on and/or around a subject's limb. The carrier may be flexible for fitting around the subject. The carrier substrate may include a single contiguous material, similar to a rubber band or may include a clasp for coupling two ends of the carrier together around the subject. In some implementations, the carrier substrate contains the light emitting elements and light detecting elements and associated electronics for performing the heart rate measurements, as well as the other operations disclosed herein.

### Pulse Oximeter

As described below, the imaging module 100 incorporating one or more multi-tap TOF demodulation pixels can be modified for use as other types of biometric sensors such as a pulse oximeter. Pulse oximeters are medical devices commonly used in the healthcare industry to measure the oxygen saturation levels in the blood non-invasively. A pulse oximeter can indicate the percent oxygen saturation and the pulse rate of the user, and can be used for many different reasons. For example, a pulse oximeter can be used to monitor an individual's pulse rate during physical exercise. An individual with a respiratory condition or a patient recovering from an illness or surgery can wear a pulse oximeter during exercise in accordance with a physician's recommendations for physical activity. Individuals also can use a pulse oximeter to monitor oxygen saturation levels to ensure adequate oxygenation, for example, during flights or during high-altitude exercising.

For pulse oximeter applications, the module 100 of FIG. 2 can be modified by the addition of a second illumination source operable to emit light at a wavelength other than the radiation emitted by the illumination source 120. The measurement principle for a pulse oximeter typically is differential, which means that the pulsation and the oxidization is measured from the difference of the power of the optical signals (e.g., the light signals sensed at the two different wavelengths). A pulse oximeter in combination with a processing unit can perform operations based on differential optical absorption spectroscopy (DOAS) by illuminating the skin and measuring changes in light absorption to obtain a photo-plethysmogram (PPG).

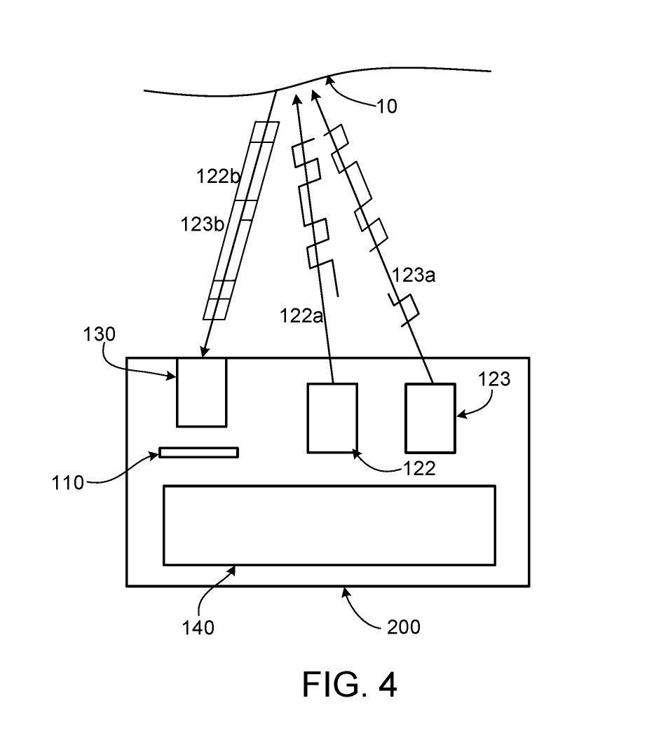

FIG. 4 shows an example of an imaging module 200 that is operable as a pulse oximeter. The module 200 includes two different illumination sources 120A, 120B. The first illumination source 120A is operable to emit light 122 at a first wavelength (e.g., in the IR part of the spectrum such as in the range 850 nm-950 nm). The second illumination source 120B is operable to emit light 123 at a different second wavelength (e.g., in the visible part of the spectrum such as at 660 nm). The module 200 includes a TOF image sensor 110 with one or more multi-tap TOF demodulation pixels, each of which can be implemented as described above in connection with FIGS. 1A and 1B. The module 200 also includes an optical system 130 and a controller 140. In a pulse oximeter implementation, each multi-tap demodulation pixel is operable to sense radiation at both the first and second wavelengths. The illumination sources 120A and 120B are temporally modulated and synchronized with the TOF image sensor 110 by the controller 140.

In some implementations, the module 200 includes a peripheral probe and a microprocessor unit including a display screen to display a waveform, oxygen saturation and pulse rate. The probe, which can contain the illumination sources 120A, 120B, can be placed in contact with the appropriate part of the individual (e.g., a finger). In operation, the emitted light penetrates and is reflected by part of a human body (e.g., an index finger). The beams of light pass through the tissues and some of the light is reflected back to the multi-tap demodulation pixels of the image sensor 110. The amount of light absorbed by blood and soft tissues depends on the concentration of hemoglobin, and the amount of light absorption at each wavelength depends on the degree of oxygenation of the hemoglobin within the tissues.

In particular, during an exposure, the illuminations sources are alternately turned on and off such that while the first illumination source 120A is turned on, the second illumination source 120B is turned off, and vice-versa. Back-reflected light 122b of the first illumination source 120A is imaged by the optical system 130 on the TOF pixel(s) of the image sensor 110, and the photo-generated charges are transferred to the first storage node(s) C1 on the TOF pixel(s), whereas back-reflected light 123b of the second illumination source 120B is captured by the same TOF pixel(s), and the photo-generated charges are transferred to the second storage node(s) C2 of the TOF pixel(s). After performing differential readout or on-pixel signal subtraction, the difference image of the two illumination sources can be measured. For example, the ratio of the different wavelength measurements (i.e., the ratio of red to infrared light absorption of pulsating components at the measuring site) can be determined by one or more processors in real-time without averaging functions. The results thus can be used to measure oxygen saturation levels in the individual's blood.

In some implementations, the imaging module 200 includes a spectral filter for each multi-tap demodulation pixel (e.g., a dual band-pass filter, operable to pass particular wavelengths of light appropriate for pulse oximetry measurements to the imaging sensor 110). Thus, for example, in some implementations, the dual band-pass filter is operable to pass light having a wavelength or range of wavelengths corresponding to visible electromagnetic radiation (e.g., 660 nm) as well as to pass light having a wavelength or range of wavelengths corresponding to infrared or near infrared radiation (e.g., 940 nm).

In some implementations, the imaging sensor also is operable, using signals sensed by the demodulation pixels, to collect distance data representative of the three-dimensionality of a scene or object, and can be further operable to collect amplitude data representative of the intensity values of the scene or object.

In some instances, a 3-tap or 4-tap pixel configuration can be used to collect signals at additional wavelengths or to collect signals when none of the illumination sources in the module is turned on.

Various modifications can be made within the spirit of the disclosure. Accordingly, other implementations are within the scope of the claims.

What is claimed is:

1. An optoelectronic module comprising:
a controller;
an illumination source operable to emit radiation of a particular wavelength toward a subject outside the module;
a TOF image sensor including one or more multi-tap demodulation pixels operable to sense radiation of the particular wavelength reflected by the subject back toward the module, each of the one or more demodulation pixels including:
a plurality of charge storage nodes, and
a read-out circuit to provide signals to the controller;
wherein the controller is operable to temporally modulate and synchronize the TOF image sensor and the illumination source, and to determine a biometric characteristic of the subject based at least in part on the signals from the one or more demodulation pixels.

2. The module of claim 1 wherein each of the one or more demodulation pixels has a 2-tap configuration.

3. The module of claim 2 wherein:
each of the one or more demodulation pixels includes:
first and second charge storage nodes; and
an in-pixel circuit to generate a differential signal based on charges stored in the pixel's first and second charge storage nodes,
wherein the read-out circuit is operable to provide the differential signal to the controller; and
wherein the controller is operable to temporally modulate and synchronize the TOF image sensor and the illumination source such that only the first storage node of each TOF pixel integrates photo-generated electrons while the illumination source is turned on, and only the second storage node of each TOF pixel integrates photo-generated electrons when the illumination source is turned off.

4. The module of claim 1 wherein the module is operable to be placed adjacent the subject such that when at least a portion of the radiation emitted by the illumination source passes into, and is reflected by, the subject, at least some of the reflected radiation is sensed by the demodulation pixels.

5. The module of claim 1 wherein the illumination source is operable to emit infra-red radiation.

6. The module of claim 1 wherein the biometric characteristic is a heart rate of the subject.

7. A method of operating an optoelectronic module, the method comprising:
emitting radiation, having a particular wavelength, from an illumination source in the module toward a subject;
sensing, by one or more multi-tap TOF demodulation pixels in the module, radiation of the particular wavelength reflected by the subject back toward the module; and
determining a biometric characteristic of the subject based at least in part on signals from the one or more demodulation pixels.

8. The method of claim 7 including temporally modulating and synchronizing the demodulation pixels and the illumination source such that only a first storage node of each respective TOF pixel integrates photo-generated electrons while the illumination source is turned on, and only a second storage node of each respective TOF pixel integrates photo-generated electrons when the illumination source is turned off.

9. The method of claim 8 wherein the biometric characteristic is a heart rate of the subject.

10. The method of claim 8 including emitting infra-red radiation from the illumination source and using the one or more demodulation pixels to sense infra-red radiation reflected by the subject.

11. The method of claim 8 including:
generating a differential signal in each of the one or more demodulation pixels;
reading out the differential signal generated by each demodulation pixel; and
determining the heart rate of the subject based at least in part on the differential signals.

12. An optoelectronic module comprising:
a controller;
a first illumination source operable to emit radiation at a first wavelength toward a subject outside the module;
a second illumination source operable to emit radiation at a second wavelength different from the first wavelength toward the subject;
an image sensor including one or more multi-tap demodulation pixels, each of which is operable to sense radiation of the first wavelength reflected by the subject back toward the module and to sense radiation of the second wavelength reflected by the subject back toward the module;
each of the one or more multi-tap demodulation pixels including:
a plurality of charge storage nodes, and
a read-out circuit to provide signals to the controller;
wherein the controller is operable to temporally modulate and synchronize the image sensor and the illumination sources, and to determine a biometric characteristic of the subject based at least in part on the signals from the demodulation pixels.

13. The module of claim 12 wherein each of the one or more demodulation pixels has a 2-tap configuration.

14. The module of claim 13 wherein:
each of one or more demodulation pixels includes:
first and second charge storage nodes; and
an in-pixel circuit to generate a differential signal based on charges stored in the pixel's first and second charge storage nodes,
wherein the read-out circuit is operable to provide the differential signal to the controller; and
wherein the controller is operable to temporally modulate and synchronize the image sensor and the illumination sources such that only one of the first or second illumination sources is turned on at a given time, such that photo-generated charges resulting from back-reflected radiation of the first illumination source are transferred to the respective first charge storage node of each of the one or more demodulation pixels, and such that photo-generated charges resulting from back-reflected radiation of the second illumination source are transferred to the respective second charge storage node of each of the one or more demodulation pixels.

15. The module of claim 12 wherein the first illumination unit is operable to emit infra-red radiation and wherein the second illumination unit is operable to emit visible radiation.

16. The module of claim 12 wherein the biometric characteristic is an oxygen saturation level of the blood of the subject.

17. The module of claim 12 including a respective dual band-pass filter over each of the one or more demodulation pixels, wherein the dual band-pass filter is operable selectively to pass radiation of the first wavelength and the second wavelength.

18. A method of operating an optoelectronic module, the method comprising:
emitting radiation of a first wavelength from a first illumination source in the module toward a subject;
sensing, by a multi-tap demodulation pixel in the module, radiation of the first wavelength reflected by the subject back toward the module;
emitting radiation of a second wavelength from a second illumination source in the module toward the subject;
sensing, by the multi-tap demodulation pixel in the module, radiation of the second wavelength reflected by the subject back toward the module; and
determining a biometric characteristic of the subject based at least in part on a signal from the multi-tap demodulation pixel.

19. The method of claim 18 including:
temporally modulating and synchronizing the demodulation pixel and the illumination sources wherein only one of the first or second illumination sources is turned on at a given time;
sensing back-reflected radiation of the first illumination source by the demodulation pixel, and transferring resulting photo-generated charges to a first charge storage node of the demodulation pixel; and
sensing back-reflected radiation of the second illumination source by the demodulation pixel, and transferring resulting photo-generated charges to a second storage node of the demodulation pixel.

20. The method of claim 19 including:
generating a differential signal based on the photo-generated charges stored, respectively in the first and second storage nodes of the demodulation pixel; and
determining the biometric characteristic of the subject based at least in part on the differential signal.

21. The method of claim 20 wherein the biometric characteristic is an oxygen saturation level of the blood of the subject.

22. The method of claim 18 including:
emitting infra-red radiation from the first illumination source; and
emitting visible radiation from the second illumination source.
